(19) United States
(12) Patent Application Publication
(10) Pub. No.: US 2003/0125358 A1
(43) Pub. Date: Jul. 3, 2003

(54) Pharmaceutical compositions containing thiazolidinedione derivatives and process for their preparation

(75) Inventors: Robin Edwin Buckingham, Harlow (GB); Michael Urquhart, Tonbridge (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/358,576

(22) Filed: Feb. 5, 2003

Related U.S. Application Data
(63) Continuation of application No. 10/049,917, filed on Feb. 19, 2002, now abandoned.
Application No. 10/049,917 is a 371 of international application No. PCT/EP00/07926, filed on Aug. 14, 2000.

(30) Foreign Application Priority Data
Aug. 17, 1999 (GB) 9919465.6
Aug. 20, 1999 (GB) 9919842.6
Aug. 25, 1999 (GB) 9920149.3

(51) Int. Cl. C07D 417/02; A61K 31/4439

(52) U.S. Cl. 514/342; 546/269.7

(57) ABSTRACT

A method for reducing post-ischaemic injury of the heart and/or improving the functional recovery of the heart following myocardial ischaemia which method comprises administration of an effective, non-toxic amount of a glucose uptake enhancer to a human or non-human mammal in need thereof

This invention relates to a novel pharmaceutical composition and to certain novel compounds, to a process for preparing such composition and compounds and to the use of such a composition and compounds in medicine.

European Patent Application, Publication Number 0306228 describes certain thiazolidinedione derivatives of formula (A):

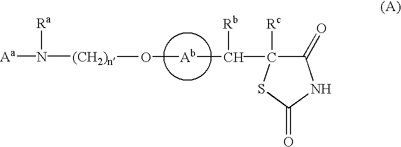

or a tautomeric form thereof and/or a pharmaceutically acceptable salt thereof, and/or a pharmaceutically acceptable solvate thereof, wherein:

A^{a} represents a substituted or unsubstituted aromatic heterocyclyl group;

R^{a} represents a hydrogen atom, an alkyl group, an acyl group, an aralkyl group, wherein the aryl moiety may be substituted or unsubstituted, or a substituted or unsubstituted aryl group;

R^{b} and R^{c} each represent hydrogen or R^{b} and R^{c} together represent a bond;

A^{b} represents a benzene ring having in total up to five substituents; and

n′ represents an integer in the range of from 2 to 6.

One particular thiazolidinedione disclosed in EP 0306228 is 5-[4-[2-(N-methyl-N-(2-pyridyl)amino)ethoxy]benzyl]thiazolidine-2,4-dione (hereinafter ‘Compound (I)’). WO94/05659 discloses certain salts of Compound (I) including the maleate salt.

Xenobiotica, 26, 6, 627-636, 1996 and J. Clin. Pharmacol, 48(3), 424-432, 1999 discloses certain metabolites of Compound (I).

It is now indicated that these compounds show good blood-glucose lowering activity and are therefore of potential use in the treatment of Type 2 diabetes or conditions associated with Type 2 diabetes.

Accordingly, the invention provides a pharmaceutical composition which composition comprises an effective non-toxic amount of a compound of formula (I):

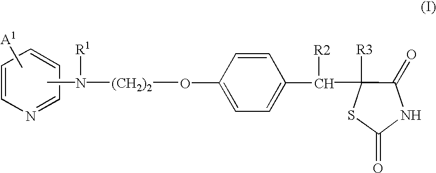

or a tautomeric form thereof and/or a pharmaceutically acceptable salt thereof, and/or a pharmaceutically acceptable solvate thereof, wherein:

A¹ represents OH or —OSO₂OH;

R¹ represents a hydrogen atom or a C₁₋₆ alkyl group; and

R² and R³ each represent hydrogen or R² and R³ together represent a bond; and a pharmaceutically acceptable carrier therefor.

In one aspect, A¹ represents OH.

Preferably, A¹ represents —OSO₂OH.

Preferably, R¹ represents a C₁₋₆alkyl group, especially a methyl group.

Preferably, R² and R³ each represent hydrogen.

Preferably, the moiety A¹-(C₅H₃N)—NR¹— represents a group of formula (a):

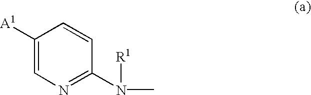

wherein A¹ and R¹ are as defined in relation to formula (I).

The invention also provides a compound of formula (I), or a tautomeric form thereof and/or a pharmaceutically acceptable salt thereof and/or a pharmaceutically acceptable solvate thereof, in solid form.

The invention also provides a compound of formula (I), or a tautomeric form thereof and/or a pharmaceutically acceptable salt thereof and/or a pharmaceutically acceptable solvate thereof, in purified form.

The invention also provides a compound of formula (I), or a tautomeric form thereof and/or a pharmaceutically acceptable salt thereof and/or a pharmaceutically acceptable solvate thereof, in crystalline form.

The invention also provides a compound of formula (I), or a tautomeric form thereof and/or a pharmaceutically acceptable salt thereof and/or a pharmaceutically acceptable solvate thereof, in pharmaceutically acceptable form.

In addition, certain of the compounds of formula (I) are novel and hence the invention also provides a compound of formula (I), or a tautomeric form thereof and/or a pharmaceutically acceptable salt thereof and/or a pharmaceutically acceptable solvate thereof; providing that formula (I) does not encompass 5-(4-{2-[(5-hydroxy-pyridin-2-yl)-methyl-amino]-ethoxy}-benzyl)-thiazolidine-2,4-dione and/or providing formula (I) does not encompass sulfuric acid mono-[6-({2-[4-(2,4-dioxo-thiazolidin-5-ylmethyl)-phenoxy]-ethyl}-methyl-amino)-pyridin-3-yl ester.

Suitable pharmaceutically acceptable salts include salts derived from pharmaceutically acceptable acids or bases.

Suitable pharmaceutically acceptable salts derived from bases include metal salts, such as for example aluminum, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-β-phenethylamine, dehydroabietylamine, N,N′-bisdehydroabietylamine, glucamine, N-methylglucamine or bases of the pyridine type such as pyridine, collidine, quinine or quinoline.

Suitable pharmaceutically acceptable acids derived from bases include acid addition salts.

Suitable acid addition salts include pharmaceutically acceptable inorganic salts such as the sulphate, nitrate, phosphate, borate, hydrochloride and hydrobromide and pharmaceutically acceptable organic acid addition salts such as acetate, tartrate, maleate, citrate, succinate, benzoate, ascorbate, methane-sulphonate, α-keto glutarate and α-glycerophosphate.

Suitable pharmaceutically acceptable solvates include hydrates.

As indicated above a compound of formula (I) may exist in one of several tautomeric forms, all of which are encompassed by the present invention. It will also be appreciated that the compounds of formula (I) can have at least one chiral carbon atom and hence can form two or more stereoisomers. The present invention encompasses all isomeric forms of the compounds of formula (I), including any stereoisomeric forms thereof, whether as individual isomers or as mixtures of isomers.

When used herein unless otherwise stipulated, “alkyl” whether present alone or as part of other groups such as alkoxy or aralkyl groups, are alkyl groups having straight or branched carbon chains, containing up to 12 carbon atoms. Suitable alkyl groups are C₁₋₆ alkyl groups e.g. methyl, ethyl, n-propyl, iso-propyl, n-butyl, iso-butyl or tert-butyl groups.

When used herein the term ‘conditions associated with diabetes’ includes those conditions associated with the pre-diabetic state, conditions associated with diabetes mellitus itself and complications associated with diabetes mellitus.

When used herein the term ‘conditions associated with the pre-diabetic state’ includes conditions such as insulin resistance, impaired fasting glucose, impaired glucose tolerance and hyperinsulinaenia.

‘Conditions associated with diabetes mellitus itself’ include hyperglycaemia, insulin resistance and obesity. Further conditions associated with diabetes mellitus itself include hypertension and cardiovascular disease, especially atherosclerosis and conditions associated with insulin resistance. Conditions associated with insulin resistance include polycystic ovarian syndrome and steroid induced insulin resistance and gestational diabetes.

‘Complications associated with diabetes mellitus’ includes renal disease, especially renal disease associated with Type 2 diabetes, neuropathy and retinopathy.

Renal diseases associated with Type 2 diabetes include nephropathy, glomerulonephritis, glomerular sclerosis, nephrotic syndrome, hypertensive nephrosclerosis and end stage renal disease.

As used herein the term ‘pharmaceutically acceptable’ embraces compounds, compositions and ingredients for both human and veterinary use: for example the term ‘pharmaceutically acceptable salt’ embraces a veterinarily acceptable salt.

The invention also provides a process for the preparation of the compounds of formula (I), which process comprises reacting a compound of formula (II):

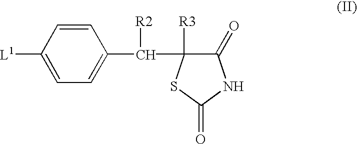

wherein R² and R³ are as defined in relation to the compounds of formula (I) and L¹ represents a leaving group, especially a halogen atom such as a fluorine atom, with a compound of formula (III):

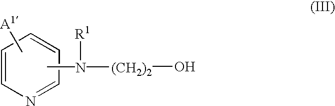

wherein R¹ is as defined in relation to formula (I) and A¹ represents A¹ as defined in relation to formula (I) or a protected form thereof; and thereafter, if required, carrying out one or more of the following optional steps:

(i) converting a compound of formula (I) into a further compound of formula (I);

(ii) removing any necessary protecting group;

(iii) preparing a pharmaceutically acceptable salt of the compound of formula (I) and/or a pharmaceutically acceptable solvate thereof.

Suitably A¹ is OH or, preferably, a protected form thereof such as a benzylated form.

Preferably the compound of formula (III) is used in an activated form, generally prepared in situ in the relevant reaction.

A suitable activated form of a compound of formula (III) is an anionic form such as a salted form and especially an alkali metal salted form, for example a potassium salt.

The activated form of the compound of formula (III) may be prepared by any appropriate conventional procedure. For example, the anionic form of the compound of formula (III) may be prepared by treating the compound of formula (III) with a base, such as a metal alkoxide, for example potassium tert-butoxide.

The reaction conditions for the reaction between the compounds of formulae (II) and (III) are generally the appropriate, conventional conditions. For example the reaction between the compound of formula (II) and a salted form of a compound of formula (III) may be carried out in an aprotic solvent, such as dimethylformamide, at any temperature providing a suitable rate of formation of the required product, conveniently at an elevated temperature for example 60° C.

The compounds of formulae (II) and (III) are known compounds or they are prepared by methods analogous to those used to prepare known compounds, for example those disclosed in EP0306228. Compound (II) wherein L¹ represents F and R² together with R³ represents a bond can also be prepared using methods disclosed by Steblyuk et al. (Fiziol. Akt. Veshchestva, 11, 97-101, 1979).

The above mentioned conversion of a compound of formula (I) into a further compound of formula (I) includes:

(a) converting one group A¹ into another group A¹;

(b) converting a compound wherein R² and R³ together represent a bond into a compound wherein R² and R³ each represent hydrogen.

The conversion of a compound of formula (I) into a further compound of formula (I) may be carried out by using any appropriate conventional procedure.

Suitable conversions (a) include the conversion of a compound wherein A¹ is OH into a compound wherein A¹is —OSO₂OH, which conversion may be carried out using pyridine/sulphur trioxide complex in a solvent such as pyridine, at any convenient temperature providing a suitable rate of formation of the required product, for example at ambient temperature.

Suitable conversions (b) may be carried out using any appropriate conventional reduction procedure, for example by use of catalytic reduction using for example a 10% palladium-on-carbon catalyst in an alkanolic solvent such as ethanol, by use of a metal/solvent system such as magnesium metal/methanol as described in Tet. Lett. 1986, 27, 2409 or by use of a metal borohydride, such as lithium borohydride (generally used in stoichiometric excess) in an aprotic solvent such as tetrahydrofuran and pyridine, typically at an elevated temperature as described in International Patent Application, Publication Number WO98/37073.

The above mentioned conversions may as appropriate be carried out on any of the intermediate compounds mentioned herein.

It will be appreciated that in any of the above mentioned reactions and conversions, any reactive group in the substrate molecule may be protected, according to conventional chemical practice. Suitable protecting groups are those used conventionally in the art. Thus, for example, suitable hydroxyl protecting groups include benzyl or trialkylsilyl groups. The methods of formation and removal of such protecting groups are those conventional methods appropriate to the molecule being protected. Thus for example a benzyloxy group may be prepared by treatment of the appropriate compound with a benzyl halide, such as benzyl bromide, and thereafter, if required, the benzyl group may be conveniently removed using mild acidic hydrolysis using, for example, hydrogen chloride in acetic acid.

As mentioned above the compounds of the invention are indicated as having useful therapeutic properties: The present invention accordingly provides a compound of formula (I), or a tautomeric form thereof and/or a pharmaceutically acceptable salt thereof and/or a pharmaceutically acceptable solvate thereof, for use as an active therapeutic substance.

Thus the present invention provides a compound of formula (I), or a tautomeric form thereof and/or a pharmaceutically acceptable salt thereof and/or a pharmaceutically acceptable solvate thereof, for use in the treatment Type 2 diabetes or conditions associated with Type 2 diabetes.

In such use the compound of formula (I), or a tautomeric form thereof and/or a pharmaceutically acceptable salt thereof and/or a pharmaceutically acceptable solvate thereof, is administered as a pharmaceutical composition of the invention also comprising a pharmaceutically acceptable carrier.

The composition of the invention is prepared by admixing a compound of formula (I), or a tautomeric form thereof and/or a pharmaceutically acceptable salt thereof and/or a pharmaceutically acceptable solvate thereof, preferably an effective non-toxic amount thereof, with a pharmaceutically acceptable carrier.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Particularly suitable compositions for oral administration are unit dosage forms such as tablets and capsules. Other fixed unit dosage forms, such as powders presented in sachets, may also be used.

In accordance with conventional pharmaceutical practice the carrier may comprise a diluent, filler, disintegrant, wetting agent, lubricant, colourant, flavourant or other conventional adjuvant.

Typical carriers include, for example, microcrystalline cellulose, starch, sodium starch glycolate, polyvinylpyrrolidone, polyvinylpolypyrrolidone, magnesium stearate or sodium lauryl sulphate.

Most suitably the composition will be formulated in unit dose form. Such unit dose will normally contain an amount of the active ingredient in the range of from 0.1 to 1000 mg, more usually 0.1 to 500 mg, and more especially 0.1 to 250 mg.

The present invention further provides a method for the treatment of Type 2 diabetes or conditions associated with Type 2 diabetes in a human or non-human mammal which comprises administering an effective, non-toxic, amount of a compound of the general formula (I), or a tautomeric form thereof and/or a pharmaceutically acceptable salt thereof and/or a pharmaceutically acceptable solvate thereof to a human or non-human mammal in need thereof.

In the treatment of Type 2 diabetes or conditions associated with Type 2 diabetes, the compound of formula (I), or a tautomeric form thereof and/or a pharmaceutically acceptable salt thereof and/or a pharmaceutically acceptable solvate thereof, may be taken in doses, such as those described above, one to six times a day in a manner such that the total daily dose for a 70 kg adult will generally be in the range of from 0.1 to 6000 mg, and more usually about 1 to 1500 mg.

In the treatment of non-human mammals, especially dogs, the active ingredient may be administered by mouth, usually once or twice a day and in an amount in the range of from about 0.025 mg/kg to 25 mg/kg, for example 0.1 mg/kg to 20 mg/kg. Similar dosage regimens are suitable for the treatment and/or prophylaxis of hyperlipidaemia in non-human mammals.

In a further aspect the present invention provides the use of a compound of formula (I), or a tautomeric form thereof and/or a pharmaceutically acceptable salt thereof and/or a pharmaceutically acceptable solvate thereof, for the manufacture of a medicament for the treatment of Type 2 diabetes or conditions associated with Type 2 diabetes.

No toxicological effects have been established for the compounds of formula (I) in the above mentioned dosage ranges.

The blood glucose lowering activity of the present compounds can be determined using standard tests such as the Oral Glucose Tolerance Test in C57b11/6 obese (ob/ob) mice, for example using methodology disclosed in EP0306228.

The following Procedures and Example illustrate the invention but do not limit it in any way.

Procedure 1: 2-[(5-Bromopyridin-2-yl)-methylamino]-ethanol.

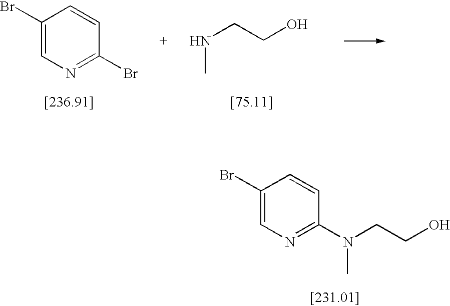

2,5-Dibromopyridine (27.93 g) and 2-(methylamino)ethanol (36.15 g) were heated under nitrogen at 150° C. for 5 hours with stirring. The reaction mixture was cooled to room temperature and added to brine (490 mL) followed by extraction with ethyl acetate (245 mL×3). The combined organic phases were washed with brine (245 mL) and dried over sodium sulphate.

The mixture was filtered and the solvent removed under reduced pressure. Toluene (100 mL×2) was added and evaporated to remove last traces of ethyl acetate. The residual oil was dried under high vacuum whereupon it crystallised.

Yield 27.23 g, 117.89 mmol, m.p. 38 to 43° C.

Infrared (nujol): νₘₐₓ 1589, 1540, 1501, 1435, 1417, 1365, 1322, 1274, 1257, 1226, 1203, 1154, 1143, 1095, 1079, 1065, 983, 954, 944, 911, 856, 811, 806, 754, 735, 722, 633, 585, 538 and 514 cm⁻¹.

PNMR (in dimethylsulphoxide, DMSO): δ_{H} 3.00 (3H, s, NCH₃), 3.53 (4H, t, NCH₂, OCH₂), 4.66 (1H, bs, OH), 6.60 (1H, d, ArH), 7.59 (2H, dd, ArH), 8.10 (1H, d, ArH).

CNMR (DMSO): δ_{c} 36.9, 51.9, 58.3, 104.8, 107.7, 139.1, 147.5, 157.0.

MS (EI, 70 eV), m/z: 230 (33.8) [M.+], 199 (100), 156 (15.0).

Procedure 2: (5-Bromo-pyridin-2yl)-[2-(tert-butyl-dimethyl-silanyloxy)-ethyl]-methyl-amine

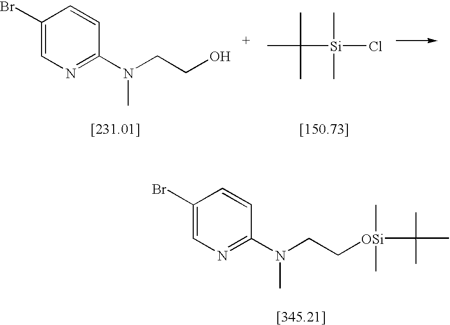

The aminoethanol from Procedure 1 (27.23 g) was dissolved in tetrahydrofuran (THF) (326 mL) with stirring under nitrogen. tert-Butyldimethylsilyl chloride (21.40 g) and imidazole (9.67 g) were added and the mixture stirred at room temperature for 18 hours. The solvent was removed under reduced pressure and the residue extracted with water (768 mL) and ethyl acetate (192 mL). The phases were separated and the aqueous re-extracted with ethyl acetate (192 mL×2). The combined organic phases were washed with water (384 mL×2), dried over sodium sulphate, and evaporated under reduced pressure to a yellow oil (weight 42.04 g)

This crude product was separated chromatographically on silica, eluting with ethyl acetate:60-80° C. petrol, 3:7 by volume. The product-rich fractions were combined and evaporated under reduced pressure to a nearly colourless oil. This was redissolved in toluene (100 mL) which was evaporated. This process was repeated once. Finally the product was dried under high vacuum to give an oil which crystallised on standing.

Yield 39.53 g, 114.51 mmol, 96.8%

Infrared (nujol): νₘₐₓ 1588, 1550, 1498, 1436, 1393, 1361, 1313, 1256, 1205, 1142, 1102, 995, 922, 836, 803, 776 and 757 cm⁻¹.

PNMR (DMSO): δ_{H} −0.03 (6H, s, Si(CH₃)₂), 0.81 (9H, s, t-BuSi), 3.00 (3H, s, NCH₃), 3.59 (2H, t, NCH₂), 3.72 (3H, t, OCH₂), 6.60 (1H, d, ArH), 7.59 (2H, dd, ArH), 8.09 (1H, d, ArH).

CNMR (DMSO): δ_{C} −5.5, 17.8, 25.7, 37.2, 51.6, 60.2, 104.9, 107.8, 139.1, 147.5, 156.9.

MS (EI, 70 eV), m/z: 344 (26.0) [M.+], 331 (7.0), 287 (85.4), 213 (34.8), 199 (100), 186 (28.0).

Procedure 3. 6-{[2-(tert-butyl-dimethyl-silanyloxy)-ethyl]-methyl-amino}-pyridin-3-ol

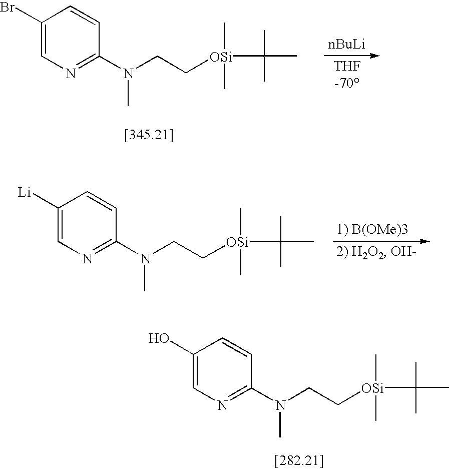

The product ex Procedure 2 (39.53 g) was dissolved in tetrahydrofuran (THF, 336 mL) with magnetic stirring under nitrogen, cooled to <−70° C. and a 2.7M solution of n-butyl lithium (52 mL) added dropwise over ca. 25 minutes ensuring that the temperature of the reaction mixture did not rise above −68° C. When the addition was complete, the reaction mixture was stirred at ca. −70° C. for a further 20 minutes.

Trimethylborane (48.3 mL) was then added to the reaction mixture over ca. 30 minutes ensuring the temperature did not rise above −65° C. Stirring at ca. −70° C. was continued for a further 20 minutes when the reaction mixture was allowed to warm up to room temperature overnight (at least 15 hours) with stirring under nitrogen.

The reaction mixture was recooled to ca. 10° C. and 2M aqueous sodium hydroxide (27 mL) added as quickly as possible whilst maintaining the temperature at ca. 10° C. (ca. 1 minute.) followed by 27.5% w/w hvdrogen peroxide (27 mL) added over 2-3 minutes while keeping the temperature <25° C. On completion of the addition, the cooling was removed and the reaction mixture stirred at ambient temperature for 2 hours.

The reaction mixture was poured into dilute hydrochloric acid (54 ml 1M hydrochloric acid+1625 ml water) and extracted with ethyl acetate (670 mL×3). The combined organic solutions were washed with water (670 mL×2) and dried over sodium sulphate. After filtration, the solvent was removed under reduced pressure to leave a dark yellow oil (35.47 g).

The oily product was chromatographed on silica, eluting with 3:7 by volume ethyl acetate/60-80° petrol. Product-rich fractions were combined, evaporated under reduced pressure and the residue dried azeotropically with toluene (100 mL×2), then under high vacuum to give a viscous yellow oil.

Yield 29.57 g, 104.78 mmol, 91.5%

Infrared (nujol): νₘₐₓ 2954, 2928, 2884, 2856, 1620, 1570, 1506, 1472, 1463, 1439, 1416, 1388, 1360, 1318, 1256, 1205, 1135, 1102, 1071, 1018, 1006, 993, 974, 938, 926, 900, 836, 809, 777, 741, 713, 695, 662 and 650 cm⁻¹.

PNMR (DMSO): δ_{H} 0.03 (6H, s, Si(CH₃)₂), 0.83 (9H, s, t-BuSi), 2.94 (3H, s, NCH₃), 3.51 (2H, t, J 6 Hz, NCH₂), 3.69 (3H, t, J 6 Hz, OCH₂), 6.46 (1H, d, J 8 Hz, ArH), 7.01 (2H, dd, J 8, 4 Hz, ArH), 7.67 (1H, d, J 4 Hz, ArH), 8.67 (1H, s, OH).

CNMR (DMSO): δ_{C} −5.5, 17.8, 25.7, 37.5, 52.1, 60.5, 106, 125.5, 134.1, 144.7, 152.6.

MS (EI, 70 eV), m/z 283 (14) [M+], 151 (100%), 136 (6), 123 (5), 110 (6).

Procedure 4. 2-[(5-Benzyloxy-pyridin-2-yl)-methyl-amino]-ethanol

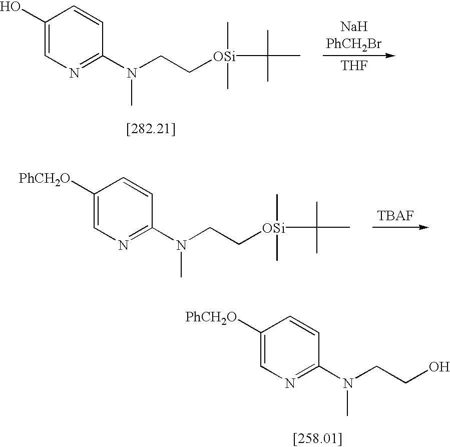

A solution of product ex procedure 3 in THF (20 mL) was added to a stirred mixture of sodium hydride (4.62 g) and benzyl bromide (12 mL) over 35 minutes. [N.B. Sodium hydride was supplied as a 60% dispersion in oil and was washed with petroleum ether and THF prior to use]. When the addition was complete, the reaction mixture was stirred at room temperature overnight.

Water (2 mL) was added followed by tetrabutylammonium fluoride hydrate (40.0 g) and the reaction mixture was stirred at room temperature for 5 hours.

Water (70 mL) was then added and the THF removed under reduced pressure. The residue was shaken with ethyl acetate (200 mL) and water (660 mL) and the phases separated. The aqueous phase was re-extracted with ethyl acetate (250 mL×2) and the combined organic phases washed with dilute brine (432 mL×2), then dried over sodium sulphate. After filtration, the solution was evaporated under reduced pressure to a dark orange oil (32.37 g).

The product was chromatographed on silica, eluting initially with ethyl acetate/60-80° petrol, 1:1 by volume, then with ethyl acetate/60-80° petrol, 7:3 by volume. The product-rich fractions were combined and evaporated under reduced pressure to give a yellow oil. This was dried by adding toluene (100 mL×2) and re-evaporating, finally drying under high vacuum. The product crystallised as a cream-coloured solid.

Yield 18.29 g, 70.89 mmol, 67.7%, m.p. 61 to 63° C.

Infrared (nujol): νₘₐₓ 1614, 1566, 1508, 1405, 1366, 1319, 1277, 1236, 1202, 1151, 1133, 1087, 1051, 1009, 972, 913, 898, 860, 824, 754, 710, 694, 643 and 541 cm⁻¹.

PNMR (DMSO): δ_{H} 2.96 (3H, s, NCH₃), 3.50 (4H, m, NCH₂, OCH₂), 4.61 (1H, t, OH), 5.03 (2H, s, OCH₂Ph), 6.56 (1H, dd, ArH), 7.28 (2H, dd, ArH), 7.31 (2H, tt, ArH), 7.38 (2H, tt, ArH), 7.42 (2H, dd, ArH), 7.86 (1H, d, ArH).

CNMR (DMSO): δ_{C} 37.2, 52.3, 58.6, 70.5, 105.9, 126.1, 127.6, 127.7, 128.3, 134.6, 137.3, 146.2, 153.9.

MS (EI, 70 eV), m/z 258 (26.8) [M.+], 227 (28.8), 167 (100), 91 (100).

Procedure 5: 5-(4-{2-[(5-Benzyloxy-pyridin-2-yl)-methyl-amino]-ethoxy}-benzylidene)-thiazolidine-2,4-dione

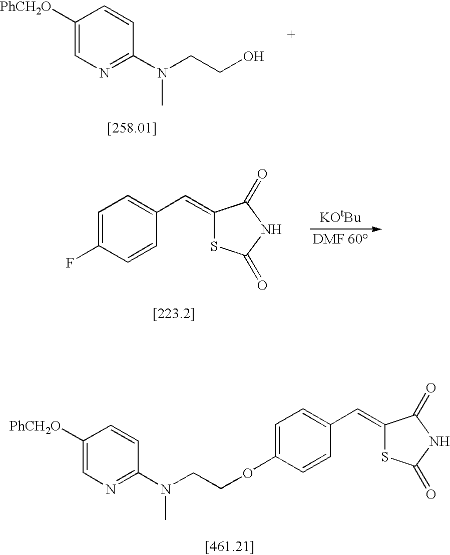

5-[(4-Fluorophenyl)methylene-2,4-thiazolidinedione (15.82 g) Steblyuk et al. (Fiziol. Akt. Veshchestva, 11, 97-101, 1979) was added to dimethylformamide (DMF, 159 mL) with magnetic stirring under nitrogen. The product ex Procedure 4 (18.29 g) was then added, followed by potassium t-butoxide (17.38 g) added in three or four portions. The resulting orange solution was heated in an oil bath at 60° C. for 4 hours when TLC showed the reaction was complete.

After cooling to ambient temperature, the reaction mixture was poured into a mixture of 1M HCl (72 mL) and water (1160 mL) with stirring to give a pale yellow suspension. The pH was 6.5. Stirring was continued for 30 minutes before the solid was filtered off, washed with water (100 mL), and sucked dry on the filter.

The paste-like damp solid was suspended in denatured ethanol (100 mL) and heated to reflux with stirring. After 30 minutes at reflux temperature the reaction mixture was allowed to cool slowly to 30° C. when cooling was applied to take the final temperature to 10° C. The solid was filtered off and washed on the filter with denatured ethanol (25 mL, 10 mL), then dried in a fan oven at 50° C. overnight.

Yield 27.46 g, 59.54 mmol, 84.0%, m.p. 186-187° C.

Infrared (nujol): νₘₐₓ 1730, 1695, 1601, 1564, 1545, 1508, 1406, 1336, 1312, 1287, 1251, 1230, 1208, 1188, 1163, 1075, 1028, 973, 896, 862, 848, 823, 813, 804, 796, 741, 716, 697, 686, 651, 631, 606, 552, 542, 520 cm⁻¹.

PNMR (DMSO): δ_{H} 3.02 (3H, s, NCH₃), 3.86 (2H, t, NCH₂), 4.19 (2H, t, OCH₂), 5.04 (2H, s, OCH₂Ph), 6.62 (1H, dd, ArH), 7.07 (2H, d, ArH), 7.31 (2H, m, ArH), 7.38 (2H, d, ArH), 7.43 (2H, t, ArH), 7.52 (2H, d, ArH), 7.67 (1H, s, ArH), 7.91 (1H, d, ArH), 12.4 (1H, bs, NH).

CNMR (DMSO): δ_{C} 36.8, 48.8, 65.8, 70.5, 106.1, 115.2, 122.1, 125.9, 126.1, 127.6, 127.7, 128.4, 130.4, 131.9, 134.7, 137.2, 146.5, 153.5, 159.9, 168.9, 169.5.

MS (EI, 70 eV), m/z 461 (0.7) [M.+], 370 (1.3), 311 (43.4), 150 (55.6), 91 (100).

Procedure 6: 5-(4-{2-[(5-Benzyloxy-pyridin-2-yl)-methyl-amino]-ethoxy}-benzyl)-thiazolidine-2,4-dione

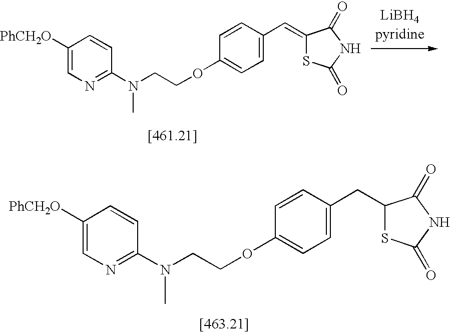

2M Lithium borohydride in THF solution (61 mL) was added to a stirred mixture of thiazolidinedione product ex Procedure 5 (26.79 g) in pyridine (47 mL) and THF (43 mL) under nitrogen over 25 minutes. After cooling to ambient, the reaction mixture was poured into a mixture of concentrated hydrochloric acid (27 mL) and ice (181 g) with stirring, maintaining the temperature <25° C. The resulting suspension was stirred at ambient temperature for 30 minutes and the pH checked. It was ca. 6.5.

The solid was filtered off and washed thoroughly with until the filtrate was colourless. The solid was azeotropically dried using toluene (100 mL) and evaporating under reduced pressure. This was repeated once. The dry residue was extracted by heating it with ethyl acetate (500 mL) to reflux temperature and filtering the hot mixture. This procedure was repeated on insoluble material with more ethyl acetate (500 mL).

The combined filtrates were evaporated to a white solid which was dried with toluene (100 mL×2) as above, then under high vacuum.

Yield 24.70 g, 53.32 mmol, 91.8%, m.p. 152-155° C.

Infrared (nujol): νₘₐₓ 1740, 1696, 1613, 1586, 1561, 1509, 1406, 1328, 1310, 1290, 1268, 1247, 1208, 1180, 1163, 1110, 1075, 1037, 1014, 975, 907, 847, 832, 810, 803, 734, 717, 695, 663, 602, 573, 548, 539, and 502 cm⁻¹.

PNMR (DMSO): δ_{H} 3.01 (4H, s, NCH₃, ArCH₂CH), 3.29 (1H, dd, ArCH₂CH), 3.82 (2H, t, NCH₂), 4.08 (2H, t, OCH₂), 4.83 (1H, dd, ArCH₂CH), 5.04 (2H, s, OCH₂Ph), 6.61 (1H, d, ArH), 6.86 (2H, d, ArH), 7.13 (2H, d, ArH), 7.31 (2H, t, ArH), 7.38 (2H, t, ArH), 7.42 (2H, d, ArH), 7.90 (1H, d, ArH), 11.80 (1H, bs, NH).

CNMR (DMSO): δ_{C} 36.3, 37.3, 48.9, 53.1, 65.4, 70.5, 106.1, 114.3, 126.1, 127.6, 127.7, 128.3, 128.6, 130.3, 134.7, 137.2, 146.5, 153.6, 157.5, 171.8, 175.9.

MS (EI, 70 eV), m/z 463 (63.5) [M.+], 372 (100), 227 (95.4), 107 (96.1), 91 (42.7).

### EXAMPLE 1

### 5-(4-{2-[(5-Hydroxy-pyridin-2-yl)-methyl-amino]-ethoxy}-benzyl)-thiazolidine-2,4-dione

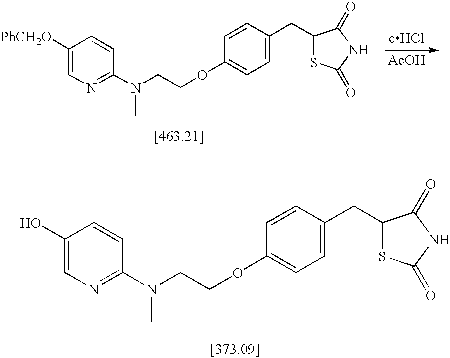

The thiazolidinedione product ex Procedure 6 (24.26 g) was dissolved in glacial acetic acid (183 mL) to which concentrated hydrochloric acid (91 mL) was added with stirring. The mixture was heated at 90° C. for 2 hours.

After cooling to ambient temperature, the acetic acid was removed under reduced pressure. The residue was vigorously stirred with ethyl acetate (535 mL) and water (535 mL) and the pH adjusted from 0.81 to 6.0 with solid sodium hydrogen carbonate. When constant pH was achieved, the phases were separated and the aqueous phase re-extracted with ethyl acetate (250 mL×2, 150 mL), keeping the solid that separated at the interface with the aqueous throughout. The combined organic phases were dried over sodium sulphate.

After filtration, the ethyl acetate was removed under reduced pressure on a rotary evaporator to leave a pale yellow foam. This was triturated with denatured ethanol (25 mL) with scratching to convert the foam to a yellow-cream solid which was filtered off and washed with IMS (10 mL total.) It was dried in a fan oven at 50° C. overnight.

Yield 13.91 g, 37.28 mmol, 71.2%, m.p. 160 to 162° C.

Infrared (nujol): νₘₐₓ 3452, 1774, 1733, 1682, 1635, 1614, 1584, 1569, 1512, 1391, 1348, 1327, 1312, 1302, 1246, 1217, 1204, 1179, 1159, 1142, 1112, 1073, 1038, 1016, 976, 942, 929, 920, 892, 851, 835, 828, 808, 733, 728, 710, 668, 605, 575, 551, 537, 526 and 505 cm⁻¹.

PNMR (DMSO): δ_{H} 2.98 (3H, s, NCH₃), 3.05 (1H, dd, ArCH₂CH), 3.29 (1H, dd, ArCH₂CH), 3.79 (2H, t, NCH₂), 4.07 (2H, t, OCH₂), 4.85 (1H, dd, ArCH₂CH), 6.53 (1H, d, ArH), 6.86 (2H, d, ArH), 7.04 (1H, dd, ArH), 7.13 (2H, d, ArH), 7.71 (1H, d, ArH), 8.74 (1H, brs, OH), 11.97 (1H, bs, NH).

CNMR (DMSO): δ_{c} 36.3, 37.5, 49.0, 53.0, 65.4, 106.2, 114.3, 125.7, 128.6, 130.3, 134.1, 145.0, 152.5, 157.5, 171.6, 175.7.

MS (EI, 70 eV), m/z 373 (3.6) [M+H], 223 (1.4), 151 (12.9), 137 (100), 107 (40.6).

### EXAMPLE 2

### Sulfuric acid mono-[6-({2-[4-(2,4-dioxo-thiazolidin-5-ylmethyl)-phenoxy]-ethyl}-methyl-amino)-pyridin-3-yl ester

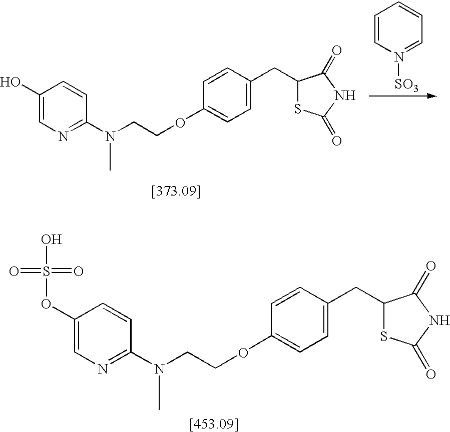

The product from Example 1 (3.40 g) and pyridine-sulphur trioxide complex (4.53 g) were dissolved in pyridine (230 mL) and stirred at room temperature under nitrogen for 26 hours. The solvent was removed under reduced pressure and toluene (70 mL) was added to the residue and re-evaporated. This was repeated with more toluene (70 mL). The residue was then treated with methanol (140 mL) and heated to reflux with stirring until dissolved and the hot solution filtered. On cooling at room temperature over 16 hours, a precipitate formed. The mixture was further cooled in a refrigerator (4-5° C.) for a further 26 hours before the solid was collected by filtration and washed with methanol (20 mL) to give crude product.

Yield 3.14 g, 6.93 mmol, 76.1%

The crude product (3.14 g) was suspended in a mixture of water (250 mL) and methanol (50 mL) and heated to reflux with stirring. The resulting yellow solution was heated for at least 30 minutes until all the solid had dissolved when it was filtered hot. The filtrate was allowed to cool to room temperature and stand for at least 20 hours. The resulting cream coloured solid was filtered off, washed with water (30 mL×2), and dried at the pump.

Yield 2.74 g, 6.05 mmol, 87.3% (recryst. recovery), m.p. 198 to 201° C.

Infrared (nujol): νₘₐₓ 3185, 3142, 3074, 3046, 1759, 1701, 1659, 1613, 1552, 1513, 1409, 1362, 1345, 1331, 1306, 1279, 1228, 1214, 1186, 1143, 1083, 1050, 1013, 1003, 974, 970, 927, 848, 816, 789, 738, 708, 686, 636, 618, 606, 573 cm⁻¹.

PNMR (DMSO): δ_{H} 3.23 (3H, s, NCH₃), 3.06 (1H, dd, ArCH₂CH), 3.26 (1H, dd, ArCH₂CH), 3.99 (2H, t, NCH₂), 4.21 (2H, t, OCH₂), 4.84 (1H, dd, ArCH₂CH), 6.84 (2H, d, ArH), 7.14 (2H, d, ArH), 7.32 (1H, dd, ArH), 7.81 (1H, d, ArH), 7.91 (1H, dd, ArH), 11.95 (1H, bs, NH).

CNMR (DMSO): δ_{c} 36.2, 38.0, 50.5, 52.9, 64.9, 112.5, 114.3, 127.8, 129.0, 130.4, 138.9, 140.9, 150.0, 157.0, 171.6, 175.6.

MS (EI, 70 eV), m/z 452 (56.3) [M−H], 372 (37.6), 229 (14.0), 222 (50.5), 116 (100).

1. A pharmaceutical composition, which composition comprises an effective non-toxic amount of a compound of formula (I): 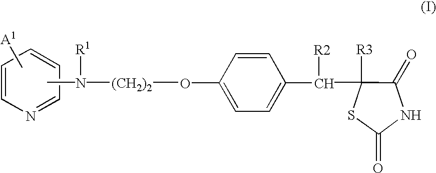
or a tautomeric form thereof and/or a pharmaceutically acceptable salt thereof, and/or a pharmaceutically acceptable solvate thereof, wherein:
A¹ represents OH or —OSO₂OH;
R¹ represents a hydrogen atom or a C₁₋₆ alkyl group; and
R² and R³ each represent hydrogen or R² and R³ together represent a bond, and a pharmaceutically acceptable carrier therefor.

2. A composition according to claim 1, wherein in the compound of formula (I) A¹ represents —OSO₂OH.

3. A composition according to claim 1 or claim 2, wherein in the compound of formula (I) R¹ represents a methyl group.

4. A composition according to any one of claims 1 to 3, wherein in the compound of formula (I) R² and R³ each represent hydrogen.

5. A composition according to any one of claims 1 to 4, wherein in the compound of formula (I) the moiety A¹-(C₅H₃N)—NR¹— represents a group of formula (a): 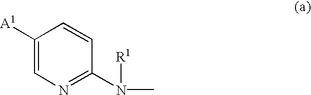
wherein A¹ and R¹ are as defined in relation to formula (I).

6. A composition according to claim 1, wherein the compound of formula (I) is 5-(4-{2-[(5-hydroxy-pyridin-2-yl)-methyl-amino]-ethoxy}-benzyl)-thiazolidine-2,4-dione or sulfuric acid mono-[6-({2-[4-(2,4-dioxo-thiazolidin-5-ylmethyl)-phenoxy]-ethyl}-methyl-amino)pyridin-3-yl ester; or a tautomeric form thereof
and/or a pharmaceutically acceptable salt thereof, and/or a pharmaceutically acceptable solvate thereof,

7. A process for the preparation of a compound of formula (I), which process comprises reacting a compound of formula (II): 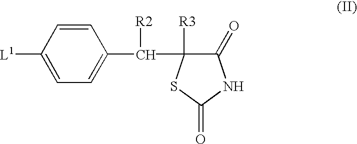
wherein R² and R³ are as defined in relation to the compounds of formula (I) and L¹ represents a leaving group with a compound of formula (III): 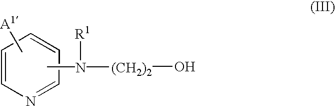
wherein R¹ is as defined in relation to formula (I) and A¹ represents A¹ as defined in relation to formula (I) or a protected form thereof; and thereafter, if required, carrying out one or more of the following optional steps:
(i) converting a compound of formula (I) into a further compound of formula (I);
(ii) removing any necessary protecting group;
(iii) preparing a pharmaceutically acceptable salt of the compound of formula (I) and/or a pharmaceutically acceptable solvate thereof.

8. A process for preparing a pharmaceutical composition according to claim 1, which process comprises admixing a compound of formula (I) as defined in claim 1, or a tautomeric form thereof and/or a pharmaceutically acceptable salt thereof and/or a pharmaceutically acceptable solvate thereof, with a pharmaceutically acceptable carrier.

9. A compound of formula (I) as defined in claim 1, or a tautomeric form thereof and/or a pharmaceutically acceptable salt thereof and/or a pharmaceutically acceptable solvate thereof, for use as an active therapeutic substance.

10. A compound of formula (I) as defined in claim 1, or a tautomeric form thereof and/or a pharmaceutically acceptable salt thereof and/or a pharmaceutically acceptable solvate thereof, for use in the treatment Type 2 diabetes or conditions associated with Type 2 diabetes.

11. A method for the treatment of Type 2 diabetes or conditions associated with Type 2 diabetes in a human or non-human mammal, which method comprises administering an effective non-toxic amount of a compound of formula (I), or a tautomeric form thereof and/or a pharmaceutically acceptable salt thereof and/or a pharmaceutically acceptable solvate thereof to a human or non-human mammal in need thereof.
